(19) United States
(12) Patent Application Publication
(10) Pub. No.: US 2008/0119493 A1
(43) Pub. Date: May 22, 2008

(54) 5,6-Dialkyl-7-Aminotriazolopyrimidines, their Preparation and their Use for Controlling Harmful Fungi, and Compositions Comprising these Compounds

(75) Inventors: Jordi Tormo i Blasco, Laudenbach (DE); Carsten Blettner, Mannheim (DE); Bernd Muller, Frankenthal (DE); Markus Gewehr, Kastellaun (DE); Wassilios Grammenos, Ludwigshafen (DE); Thomas Grote, Wachenheim (DE); Joachim Rheinheimer, Ludwigshafen (DE); Peter Schafer, Ottersheim (DE); Frank Schieweck, Hessheim (DE); Anja Schwogler, Mannheim (DE); Oliver Wagner, Neustadt (DE); Matthias Niedenbruck, Limburgerhof (DE); Maria Scherer, Godramstein (DE); Siegfried Strathmann, Limburgerhof (DE); Ulrich Schofl, Bruhl (DE); Reinhard Stierl, Freinsheim (DE); Udo Hunger, Mainz (DE)

Correspondence Address: BIRCH STEWART KOLASCH & BIRCH, PO BOX 747, FALLS CHURCH, VA, 22040-0747, US

(73) Assignee: BASF Aktiengesellschaft, Ludwigshafen (DE)

(21) Appl. No.: 10/590,326

(22) PCT Filed: Mar. 8, 2005
(86) PCT No.: PCT/EP05/02424

§ 371 Date: Aug. 23, 2006

(30) Foreign Application Priority Data
Mar. 10, 2004 (DE) 102004012019.6
Mar. 10, 2004 (DE) 102004012021.8

(51) Int. Cl. A01N 43/90; C07D 487/04; A01P 3/00

(52) U.S. Cl. 514/259.31; 544/263

(57) ABSTRACT

The invention relates to 5,6-dialkyl-7-amino-triazolopyrimidines of formula (I), in which the substituents are defined as follows: R¹ represents alkyl, alkoxyalkyl, alkenyl or alkynyl; R² represents alkyl, alkoxyalkyl, alkenyl or alkynyl, R¹ and/or R² being substituted according to the description. The invention also relates to a method for producing said compounds, to agents containing the latter and to their use for controlling plant-pathogenic fungi.

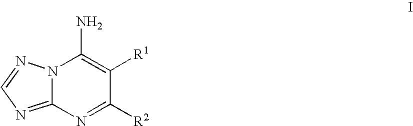

The present invention relates to 5,6-dialkyl-7-aminotriazolopyrimidines of the formula I

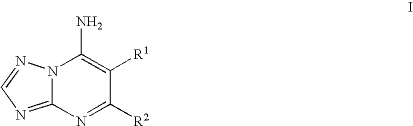

in which the substituents are as defined below:
R¹ is C₂-C₁₂-alkenyl or C₂-C₁₂-alkynyl, where the carbon chains are unsubstituted or carry one to three identical or different groups R^{a} and/or R^{b};
   or
   C₁-C₁₄-alkyl, C₁-C₁₂-alkoxy-C₁-C₁₂-alkyl, C₁-C₆-alkoxy-C₂-C₁₂-alkenyl or C₁-C₆-alkoxy-C₂-C₁₂-alkynyl, where the carbon chains carry one to three identical or different groups R^{a};
   R^{a} is halogen, cyano, nitro, hydroxyl, C₁-C₆-alkylthio, C₃-C₁₂-alkenyloxy, C₃-C₁₂-alkynyloxy, NR¹¹R¹², or
      C₃-C₆-cycloalkyl which may carry one to four identical or different groups R^{b};
      R^{b} is C₁-C₄-alkyl, cyano, nitro, hydroxyl, C₁-C₆-alkoxy, C₁-C₆-alkylthio, C₃-C₆-alkenyloxy, C₃-C₆-alkynyloxy and NR¹¹R¹²
         R¹¹, R¹² are hydrogen or C₁-C₆-Alkyl;
      where the carbon chains of the groups R^{a} for their part may be halogenated;
R² is C₁-C₁₂-alkyl, C₂-C₁₂-alkenyl or C₂-C₁₂-alkynyl, where the carbon chains may be substituted by one to three groups R^{c}:
   R^{c} is cyano, nitro, hydroxyl, NR¹¹R¹²; or C₃-C₆-cycloalkyl which may carry one to four identical or different groups C₁-C₄-alkyl, halogen, cyano, nitro, hydroxyl, C₁-C₆-alkoxy, C₁-C₆-alkylthio, C₃-C₆-alkenyloxy, C₃-C₆-alkynyloxy
      or NR¹¹R¹².

Moreover, the invention relates to processes for preparing these compounds, to compositions comprising them and to their use for controlling phytopathogenic harmful fungi.

5,6-Dialkyl-7-aminotriazolopyrimidines are proposed in a general manner in GB 1 148 629. Individual fungicidally active 5,6-dialkyl-7-aminotriazolopyrimidines are known from EP-A 141 317. However, in many cases their activity is unsatisfactory. Based on this, it is an object of the present invention to provide compounds having improved activity and/or a wider activity spectrum.

We have found that this object is achieved by the definitions defined at the outset. Furthermore, we have found processes and intermediates for their preparation, compositions comprising them and methods for controlling harmful fungi using the compounds I.

The compounds of the formula I differ from those in the abovementioned publications by the specific embodiment of the substituent in the 6-position of the triazolopyrimidine skeleton, which is a haloalkyl group or an unsaturated aliphatic group.

Compared to the known compounds, the compounds of the formula I are more effective against harmful fungi.

The compounds according to the invention can be obtained by different routes. Advantageously, the compounds according to the invention are obtained by converting substituted β-ketoesters of the formula II with 3-amino-1,2,4-triazole of the formula III to give 7-hydroxytriazolopyrimidines of the formula IV. The groups R¹ and R² in formulae II and IV are as defined for formula I and the group R in formula II is C₁-C₄-alkyl; for practical reasons, preference is given here to methyl, ethyl or propyl.

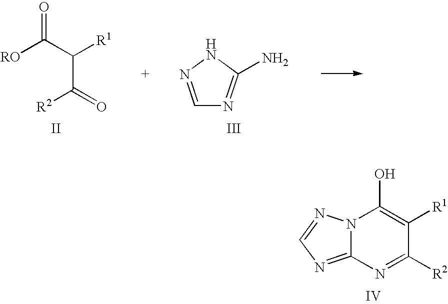

The reaction of the substituted β-ketoesters of the formula II with the aminoazoles of the formula III can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as alkali metal and alkaline earth metal hydroxides, alkali metal and alkaline earth metal oxides, alkali metal and alkaline earth metal hydrides, alkali metal amides, alkali metal and alkaline earth metal carbonates and also alkali metal bicarbonates, organometallic compounds, in particular alkali metal alkyls, alkylmagnesium halides and also alkali metal and alkaline earth metal alkoxides and dimethoxymagnesium, moreover organic bases, for example tertiary amines, such as trimethylamine, triethylamine, triisopropylethylamine, tributylamine and N-methylpiperidine, N-methylmorpholine, pyridine, substituted pyridines, such as collidine, lutidine and 4-dimethylaminopyridine, and also bicyclic amines and mixtures of these solvents with water. Suitable catalysts are bases, such as those mentioned above, or acids, such as sulfonic acids or mineral acids. With particular preference, the reaction is carried out in the absence of a solvent or in chlorobenzene, xylene, dimethyl sulfoxide or N-methylpyrrolidone. Particularly preferred bases are tertiary amines, such as triisopropylamine, tributylamine, N-methylmorpholine or N-methylpiperidine. The temperatures are from 50 to 300° C., preferably from 50 to 180° C., if the reaction is carried out in solution [cf. EP-A 770 615; Adv. Het. Chem. 57 (1993), 81ff].

The bases are generally employed in catalytic amounts; however, they can also be employed in equimolar amounts, in excess or, if appropriate, as solvent.

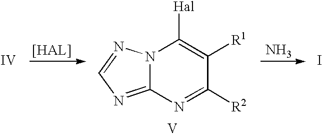

In most cases, the resulting condensates of the formula IV precipitate from the reaction solutions in pure form and, after washing with the same solvent or with water and subsequent drying they are reacted with halogenating agents, in particular chlorinating or brominating agents, to give the compounds of the formula V in which Hal is chlorine or bromine, in particular chlorine. The reaction is preferably carried out using chlorinating agents such as phosphorus oxychloride, thionyl chloride or sulfuvyl chloride at from 50° C. to 150° C., preferably in excess phosphorus oxytrichloride at reflux temperature. After evaporation of excess phosphorus oxytrichloride, the residue is treated with ice-water, if appropriate with addition of a water-immiscible solvent. In most cases, the chlorinated product isolated from the dried organic phase, if appropriate after evaporation of the inert solvent, is very pure and is subsequently reacted with ammonia in inert solvents at from 100° C. to 200° C. to give the 7-amino-triazolo[1,5-a]pyrimidines. This reaction is preferably carried out using a 1- to 10-molar excess of ammonia, under a pressure of from 1 to 100 bar.

The novel 7-aminoazolo[1,5-a]pyrimidines are, if appropriate after evaporation of the solvent, isolated as crystalline compounds, by digestion in water.

The β-ketoesters of the formula II can be prepared as described in Organic Synthesis Coll. Vol. 1, p. 248, and/or they are commercially available.

Alternatively, the novel compounds of the formula I can be obtained by reacting substituted acyl cyanides of the formula VI in which R¹ and R² are as defined above with 3-amino-1,2,4-triazole of the formula III.

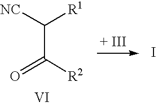

The reaction can be carried out in the presence or absence of solvents. It is advantageous to use solvents to which the starting materials are substantially inert and in which they are completely or partially soluble. Suitable solvents are in particular alcohols, such as ethanol, propanols, butanols, glycols or glycol monoethers, diethylene glycols or their monoethers, aromatic hydrocarbons, such as toluene, benzene or mesitylene, amides, such as dimethylformamide, diethylformamide, dibutylformamide, N,N-dimethylacetamide, lower alkanoic acids, such as formic acid, acetic acid, propionic acid, or bases, such as those mentioned above, and mixtures of these solvents with water. The reaction temperatures are from 50 to 300° C., preferably from 50 to 150° C., if the reaction is carried out in solution.

Some of the substituted alkyl cyanides of the formula VI required for preparing the 7-aminoazolo[1,5-a]pyrimidines are known, or they can be prepared by known methods from alkyl cyanides and carboxylic acid esters using strong bases, for example alkali metal hydrides, alkali metal alcoholates, alkali metal amides or metal alkyls (cf.: J. Amer. Chem. Soc. 73, (1951), p. 3766).

Compounds of the formula I in which R¹ is C₁-C₁₄-haloalkyl, C₁-C₁₂-haloalkoxy-C₁-C₁₂-alkyl, C₁-C₁₂-alkoxy-C₁-C₁₂-haloalkyl, C₂-C₁₂-haloalkenyl or C₂-C₁₂-haloalkynyl can be obtained advantageously by halogenating corresponding triazolopyrimidines of the formula VII:

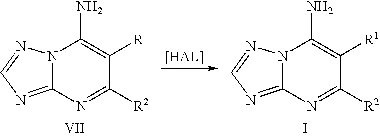

In the formula VII, R is C₁-C₁₄-alkyl, C₁-C₁₂-alkoxy-C₁-C₁₂-alkyl, C₂-C₁₂-alkenyl, C₂-C₁₂-alkynyl, where the carbon chains may carry one to three groups R^{a}.

The halogenation is usually carried out at temperatures of from 0° C. to 200° C., preferably from 20° C. to 110° C., in an inert organic solvent in the presence of a free-radical initiator (for example dibenzoyl peroxide or azobisisobutyronitrile or under UV irradiation, for example with an Hg vapor lamp) or an acid [cf. Synthetic Reagents, volume 2, pp. 1-63, Wiley, New York (1974)].

The reaction partners are generally reacted with one another in equimolar amounts. In terms of yield, it may be advantageous to employ an excess of halogenating agent, based on VII.

Suitable halogenating agents are, for example, elemental halogens (for example Cl₂, Br₂, I₂), N-bromosuccinimide, N-chlorosuccinimide or dibromodimethylhydrantoin. The halogenating agents are generally employed in equimolar amounts, in excess or, if appropriate, as solvent.

Alternatively, compounds of the formula I, in which R¹ is C₁-C₁₄-haloalkyl, C₂-C₁₂-haloalkenyl or C₂-C₁₂-haloalkynyl can be obtained by ether cleavage of corresponding triazolopyrimidines of the formula VIIa:

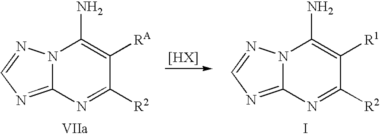

In the formula VIIa, R^{A} is C₁-C₁₄-alkyl, C₂-C₁₂-alkenyl or C₂-C₁₂-alkynyl, where the groups R^{A} are substituted by hydroxyl or alkoxycarbonyl groups. By heating the compounds VIIa in the presence of mineral acids [HX], such as hydrochloric acid or hydrobromic acid, or nitric acid, the compounds I are obtained [cf. Organikum, 15th edition, p. 237 ff., VEB Deutscher Verlag der Wissenschaften, Berlin 1981].

Some of the triazolopyrimidines of the formulae VII and VIIa required for preparing the compounds I described above are known, or they can be prepared by known methods [cf. EP-A 141 317].

If individual compounds I can not be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example during the treatment of plants within the treated plants, or in the harmful fungus to be controlled.

In the definitions of symbols given above, collective terms were used which are generally representative of the following substituents:
halogen: fluorine, chlorine, bromine and iodine in particular fluorine or chlorine;
alkyl: saturated straight-chain or branched hydrocarbon radicals having 1 to 4, 6, 8 or 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated straight-chain or branched hydrocarbon radicals having 2 to 4, 6, 8 or 10 carbon atoms and one or two double bonds in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkoxyalkyl: a saturated straight-chain or mono-, di- or tribranched hydrocarbon chain which is interrupted by an oxygen atom, for example C₅-C₁₂-alkoxyalkyl: a hydrocarbon chain as described above having 5 to 12 carbon atoms which may be interrupted by an oxygen in any position, such as propoxyethyl, butoxyethyl, pentoxyethyl, hexyloxyethyl, heptyloxyethyl, octyloxyethyl, nonyloxyethyl, 3-(3-ethylhexyloxy)ethyl, 3-(2,4,4-tri-methylpentyloxy)ethyl, 3-(1-ethyl-3-methylbutoxy)ethyl, ethoxypropyl, propoxypropyl, butoxypropyl, pentoxypropyl, hexyloxypropyl, heptyloxypropyl, octyloxypropyl, nonyloxypropyl, 3-(3-ethylhexyloxy)propyl, 3-(2,4,4-trimethylpentyloxy)propyl, 3-(1-ethyl-3-methyl butoxy)propyl, ethoxybutyl, propoxybutyi, butoxybutyl, pentoxybutyl, hexyloxybutyl, heptyloxybutyl, octyloxybutyl, nonyloxybutyl, 3-(3-ethylhexyloxy)butyl, 3-(2,4,4-trimethylpentyloxy)butyl, 3-(1-ethyl-3-methylbutoxy)butyl, methoxypentyl, ethoxypentyl, propoxypentyl, butoxypentyl, pentoxypentyl, hexyloxypentyl, heptyloxypentyl, 3-(3-methylhexyloxy)pentyl, 3-(2,4-dimethylpentyloxy)pentyl, 3-(1-ethyl-3-methylbutoxy)pentyl;
haloalkenyl: unsaturated straight-chain or branched hydrocarbon radicals having 2 to 10 carbon atoms and one or two double bonds in any position (as mentioned above), where some or all of the hydrogen atoms in these groups may be replaced by halogen atoms as mentioned above, in particular by fluorine, chlorine and bromine;
alkynyl: straight-chain or branched hydrocarbon groups having 2 to 4, 6, 8 or 10 carbon atoms and one or two triple bonds in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 carbon ring members, such as cyclopropyl, cyclobutyl, cyclopentyl and cyclohexyl;

The scope of the present invention includes the (R)- and (S)-isomers and the racemates of compounds of the formula I having chiral centers.

With a view to the intended use of the triazolopyrimidines of the formula I, particular preference is given to the following meanings of the substituents, in each case on their own or in combination:

Preference is given to compounds I in which the group R¹ has at most 9 carbon atoms.

Likewise, preference is given to compounds of the formula I in which R¹ is a straight-chain or mono-, di-, tri- or polybranched haloalkyl group.

If R¹ is haloalkyl, the halogenation is preferably at the terminal carbon. Preference is given to monohaloalkyl groups.

In one embodiment of the compounds I according to the invention, R¹ is C₁-C₁₄-haloalkyl, C₁-C₁₂-haloalkoxy-C₁-C₁₂-alkyl, C₁-C₁₂-alkoxy-C₁-C₁₂-haloalkyl, C₂-C₁₂-haloalkenyl or C₂-C₁₂-haloalkynyl, the groups having one or two halogen atoms. C₁-C₉-haloalkoxypropyl and C₁-C₉-alkoxyhalopropyl groups ae preferred here.

In another embodiment of the compounds I, R¹ is a group C₁-C₁₄-haloalkyl, C₁-C₁₂-haloalkoxy-C₁-C₁₂-alkyl, C₁-C₁₂-alkoxy-C₁-C₁₂-haloalkyl, C₂-C₁₂-haloalkenyl or C₂-C₁₂-haloalkynyl, which groups contain a halogen atom at the α carbon atom.

In addition, preference is given to compounds of the formula I in which R¹ is a group (CH₂)ₙCH₂Cl, (CH₂)ₙCH₂Br, CH(CH₃)(CH₂)ₘCH₂Cl, CH(CH₃)(CH₂)ₘCH₂Br, (CH₂)ₙCF₃ or CH(CH₃)(CH₂)ₘCF₃, where n is a number from 0 to 13 and m is a number from 0 to 11.

Particular preference is given to compounds I in which R¹ is chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1,1-trifluoroprop-2-yl, 1-chloropropyl, 1-fluoropropyl, 3-chloropropyl, 3-fluoropropyl, 3,3,3-trifluoropropyl, 1-chlorobutyl, 1-fluorobutyl, 4-chlorobutyl, 4-fluorobutyl, 4,4,4-trifluorobutyl, 1-chloropentyl, 1-fluoropentyl, 5,5,5-trifluoropentyl, 5-chloropentyl, 5-fluoropentyl, 1-chlorohexyl, 1-fluorohexyl, 6-chlorohexyl, 6-fluorohexyl, 6,6,6-trifluorohexyl, 1-chloroheptyl, 1-fluoroheptyl, 7-chloroheptyl, 7-fluoroheptyl, 7,7,7-trifluoroheptyl, 1-chlorooctyl, 1-fluorooctyl, 8-fluorooctyl, 8,8,8-trifluorooctyl, 1-chlorononyl, 1-fluorononyl, 9-fluorononyl, 9,9,9-trifluorononyl, 9-chlorononyl, 1-fluorodecyl, 1-chlorodecyl, 10-fluorodecyl, 10,10,10-trifluorodecyl, 10-chlorodecyl, 1-chloroundecyl, 1-fluoroundecyl, 11-chloroundecyl, 11-fluoroundecyl, 11,11,11-trifluoroundecyl, 1-chlorododecyl, 1-fluorododecyl, 12-chlorododecyl, 12-fluorododecyl or 12,12,12-trifluorododecyl.

In a further embodiment of the compounds I, R¹ is C₂-C₁₂-alkenyl or C₂-C₁₂-Alkynyl, where the hydrocarbon chains are unsubstituted or carry one to three identical or different groups R^{a} and/or R^{b}.

In a preferred embodiment of the compounds of the formula I the group R^{a} is absent.

Particular preference is given to compounds I in which the carbon chains of R¹ and R² together do not have more than 14 carbon atoms.

In one embodiment of the compounds I according to the invention, R² is methyl, ethyl, isopropyl, n-propyl or n-butyl, preferably methyl, ethyl, isopropyl or n-propyl, in particular methyl or ethyl.

Halogen atoms in the groups R¹ are preferably located at the α or Ω carbon atom.

Cyano groups in R¹ and/or R² are preferably located at the terminal carbon atom.

In a further preferred embodiment of the compounds of the formula I the group R^{b} is absent.

In particular with a view to their use, preference is given to the compounds I compiled in the tables below. Moreover, the groups mentioned for a substituent in the tables are per se, independently of the combination in which they are mentioned, a particularly preferred embodiment of the substituent in question.

### Table 1

Compounds of the formula I in which R¹ for each compound corresponds to one row of Table A and R² is methyl

### Table 2

Compounds of the formula I in which R¹ for each compound corresponds to one row of Table A and R² is ethyl

### Table 3

Compounds of the formula I in which R¹ for each compound corresponds to one row of Table A and R² is n-propyl

### Table 4

Compounds of the formula I in which R¹ for each compound corresponds to one row of Table A and R² is isopropyl

### Table 5

Compounds of the formula I in which R¹ for each compound corresponds to one row of Table A and R² is n-butyl

TABLE A

| No. | R¹ |
|---|---|
| A-1 | CH₂F |
| A-2 | CH₂Cl |
| A-3 | CH₂Br |
| A-4 | CHF₂ |
| A-5 | CHCl₂ |
| A-6 | CF₃ |
| A-7 | CCl₃ |
| A-8 | CHFCH₃ |
| A-9 | CHClCH₃ |
| A-10 | CH₂CH₂F |
| A-11 | CH₂CH₂Cl |
| A-12 | CH₂CH₂Br |
| A-13 | CCl₂CH₃ |
| A-14 | CF₂CH₃ |
| A-15 | CH₂CHF₂ |
| A-16 | CH₂CHCl₂ |
| A-17 | CH₂CF₃ |
| A-18 | CH₂CCl₃ |
| A-19 | CF₂CF₃ |
| A-20 | CCl₂CCl₃ |
| A-21 | CHFCH₂CH₃ |
| A-22 | CHClCH₂CH₃ |
| A-23 | CH₂CHFCH₃ |
| A-24 | CH₂CHClCH₃ |
| A-25 | CH₂CH₂CH₂F |
| A-26 | CH₂CH₂CH₂Cl |
| A-27 | CH₂CH₂CH₂Br |
| A-28 | CCl₂CH₂CH₃ |
| A-29 | CF₂CH₂CH₃ |
| A-30 | CH₂CH₂CHF₂ |
| A-31 | CH₂CH₂CHCl₂ |
| A-32 | CH₂CH₂CF₃ |
| A-33 | CH₂CH₂CCl₃ |
| A-34 | CF₂CF₂CF₃ |
| A-35 | CCl₂CCl₂CCl₃ |
| A-36 | CH(CH₃)CF₃ |
| A-37 | CH(CH₃)CH₂F |
| A-38 | CH(CH₃)CH₂Cl |
| A-39 | CH(CH₃)CH₂Br |
| A-40 | CH(CH₃)CHF₂ |
| A-41 | CH(CH₃)CHCl₂ |
| A-42 | CH(CH₂F)₂ |
| A-43 | CH(CH₂Cl)₂ |
| A-44 | CH(CH₂Br)₂ |
| A-45 | CH(CHF₂)₂ |
| A-46 | CH(CHCl₂)₂ |
| A-47 | CHFCH₂CH₂CH₃ |
| A-48 | CHClCH₂CH₂CH₃ |
| A-49 | CH₂CHFCH₂CH₃ |
| A-50 | CH₂CHClCH₂CH₃ |
| A-51 | CH₂CH₂CHFCH₃ |
| A-52 | CH₂CH₂CHClCH₃ |
| A-53 | CH₂CH₂CH₂CH₂F |
| A-54 | CH₂CH₂CH₂CH₂Cl |
| A-55 | CH₂CH₂CH₂CH₂Br |
| A-56 | CCl₂CH₂CH₂CH₃ |
| A-57 | CF₂CH₂CH₂CH₃ |
| A-58 | CH₂CH₂CH₂CHF₂ |
| A-59 | CH₂CH₂CH₂CHCl₂ |
| A-60 | CH₂CH₂CH₂CF₃ |
| A-61 | CH₂CH₂CH₂CCl₃ |
| A-62 | CF₂CF₂CF₂CF₃ |
| A-63 | CCl₂CCl₂CCl₂CCl₃ |
| A-64 | CH(CH₃)CH₂CH₂F |
| A-65 | CH(CH₃)CH₂CH₂Cl |
| A-66 | CH(CH₃)CH₂CH₂Br |
| A-67 | CH(CH₃)CH₂CF₃ |
| A-68 | CHFCH₂CH₂CH₂CH₃ |
| A-69 | CHClCH₂CH₂CH₂CH₃ |
| A-70 | CH₂CHFCH₂CH₂CH₃ |
| A-71 | CH₂CHClCH₂CH₂CH₃ |
| A-72 | CH₂CH₂CHFCH₂CH₃ |
| A-73 | CH₂CH₂CHClCH₂CH₃ |
| A-74 | CH₂CH₂CH₂CHFCH₃ |
| A-75 | CH₂CH₂CH₂CHClCH₃ |
| A-76 | CH₂CH₂CH₂CH₂CH₂F |
| A-77 | CH₂CH₂CH₂CH₂CH₂Cl |
| A-78 | CH₂CH₂CH₂CH₂CH₂Br |
| A-79 | CCl₂CH₂CH₂CH₂CH₃ |
| A-80 | CF₂CH₂CH₂CH₂CH₃ |
| A-81 | CH₂CH₂CH₂CH₂CHF₂ |
| A-82 | CH₂CH₂CH₂CH₂CHCl₂ |
| A-83 | CH₂CH₂CH₂CH₂CF₃ |
| A-84 | CH₂CH₂CH₂CH₂CCl₃ |
| A-85 | CF₂CF₂CF₂CF₂CF₃ |
| A-86 | CCl₂CCl₂CCl₂CCl₂CCl₃ |
| A-87 | CH(CH₃)CH₂CH₂CH₂F |
| A-88 | CH(CH₃)CH₂CH₂CH₂Cl |
| A-89 | CH(CH₃)CH₂CH₂CH₂Br |
| A-90 | CH(CH₃)CH₂CH₂CF₃ |
| A-91 | CHFCH₂CH₂CH₂CH₂CH₃ |
| A-92 | CHClCH₂CH₂CH₂CH₂CH₃ |
| A-93 | CH₂CHFCH₂CH₂CH₂CH₃ |
| A-94 | CH₂CHClCH₂CH₂CH₂CH₃ |
| A-95 | CH₂CH₂CHFCH₂CH₂CH₃ |
| A-96 | CH₂CH₂CHClCH₂CH₂CH₃ |
| A-97 | CH₂CH₂CH₂CHFCH₂CH₃ |
| A-98 | CH₂CH₂CH₂CHClCH₂CH₃ |
| A-99 | CH₂CH₂CH₂CH₂CHFCH₃ |
| A-100 | CH₂CH₂CH₂CH₂CHClCH₃ |
| A-101 | CH₂CH₂CH₂CH₂CH₂CH₂F |
| A-102 | CH₂CH₂CH₂CH₂CH₂CH₂Cl |
| A-103 | CH₂CH₂CH₂CH₂CH₂CH₂Br |
| A-104 | CCl₂CH₂CH₂CH₂CH₂CH₃ |
| A-105 | CF₂CH₂CH₂CH₂CH₂CH₃ |
| A-106 | CH₂CH₂CH₂CH₂CH₂CHF₂ |
| A-107 | CH₂CH₂CH₂CH₂CH₂CHCl₂ |
| A-108 | CH₂CH₂CH₂CH₂CH₂CF₃ |
| A-109 | CH₂CH₂CH₂CH₂CH₂CCl₃ |
| A-110 | CF₂CF₂CF₂CF₂CF₂CF₃ |
| A-111 | CCl₂CCl₂CCl₂CCl₂CCl₂CCl₃ |
| A-112 | CH(CH₃)CH₂CH₂CH₂CH₂F |
| A-113 | CH(CH₃)CH₂CH₂CH₂CH₂Cl |
| A-114 | CH(CH₃)CH₂CH₂CH₂CH₂Br |
| A-115 | CH(CH₃)CH₂CH₂CH₂CF₃ |
| A-116 | CHFCH₂CH₂CH₂CH₂CH₂CH₃ |
| A-117 | CHClCH₂CH₂CH₂CH₂CH₂CH₃ |
| A-118 | CH₂CHFCH₂CH₂CH₂CH₂CH₃ |
| A-119 | CH₂CHClCH₂CH₂CH₂CH₂CH₃ |
| A-120 | CH₂CH₂CH₂CHFCH₂CH₂CH₃ |
| A-121 | CH₂CH₂CH₂CHClCH₂CH₂CH₃ |
| A-122 | CH₂CH₂CH₂CH₂CHFCH₂CH₃ |
| A-123 | CH₂CH₂CH₂CH₂CHClCH₂CH₃ |
| A-124 | CH₂CH₂CH₂CH₂CH₂CHFCH₃ |
| A-125 | CH₂CH₂CH₂CH₂CH₂CHClCH₃ |
| A-126 | CH₂CH₂CH₂CH₂CH₂CH₂CH₂F |
| A-127 | CH₂CH₂CH₂CH₂CH₂CH₂CH₂Cl |
| A-128 | CH₂CH₂CH₂CH₂CH₂CH₂CH₂Br |
| A-129 | CCl₂CH₂CH₂CH₂CH₂CH₂CH₃ |
| A-130 | CF₂CH₂CH₂CH₂CH₂CH₂CH₃ |
| A-131 | CH₂CH₂CH₂CH₂CH₂CH₂CHF₂ |
| A-132 | CH₂CH₂CH₂CH₂CH₂CH₂CHCl₂ |
| A-133 | CH₂CH₂CH₂CH₂CH₂CH₂CF₃ |
| A-134 | CH₂CH₂CH₂CH₂CH₂CH₂CCl₃ |
| A-135 | CF₂CF₂CF₂CF₂CF₂CF₂CF₃ |
| A-136 | CCl₂CCl₂CCl₂CCl₂CCl₂CCl₂CCl₃ |
| A-137 | CH(CH₃)CH₂CH₂CH₂CH₂CH₂F |
| A-138 | CH(CH₃)CH₂CH₂CH₂CH₂CH₂Cl |
| A-139 | CH(CH₃)CH₂CH₂CH₂CH₂CH₂Br |
| A-140 | CH(CH₃)CH₂CH₂CH₂CH₂CF₃ |
| A-141 | CHFCH₂CH₂CH₂CH₂CH₂CH₂CH₃ |
| A-142 | CHClCH₂CH₂CH₂CH₂CH₂CH₂CH₃ |
| A-143 | CH₂CHFCH₂CH₂CH₂CH₂CH₂CH₃ |
| A-144 | CH₂CHClCH₂CH₂CH₂CH₂CH₂CH₃ |
| A-145 | CH₂CH₂CHFCH₂CH₂CH₂CH₂CH₃ |
| A-146 | CH₂CH₂CHClCH₂CH₂CH₂CH₂CH₃ |
| A-147 | CH₂CH₂CH₂CH₂CHFCH₂CH₂CH₃ |
| A-148 | CH₂CH₂CH₂CH₂CHClCH₂CH₂CH₃ |
| A-149 | CH₂CH₂CH₂CH₂CH₂CHFCH₂CH₃ |
| A-150 | CH₂CH₂CH₂CH₂CH₂CHClCH₂CH₃ |
| A-151 | CH₂CH₂CH₂CH₂CH₂CH₂CHFCH₃ |
| A-152 | CH₂CH₂CH₂CH₂CH₂CH₂CHClCH₃ |
| A-153 | CH₂CH₂CH₂CH₂CH₂CH₂CH₂CH₂F |
| A-154 | CH₂CH₂CH₂CH₂CH₂CH₂CH₂CH₂Cl |
| A-155 | CH₂CH₂CH₂CH₂CH₂CH₂CH₂CH₂Br |
| A-156 | CCl₂CH₂CH₂CH₂CH₂CH₂CH₂CH₃ |
| A-157 | CF₂CH₂CH₂CH₂CH₂CH₂CH₂CH₃ |
| A-158 | CH₂CH₂CH₂CH₂CH₂CH₂CH₂CHF₂ |
| A-159 | CH₂CH₂CH₂CH₂CH₂CH₂CH₂CHCl₂ |
| A-160 | CH₂CH₂CH₂CH₂CH₂CH₂CH₂CF₃ |
| A-161 | CH₂CH₂CH₂CH₂CH₂CH₂CH₂CCl₃ |
| A-162 | CF₂CF₂CF₂CF₂CF₂CF₂CF₂CF₃ |
| A-163 | CCl₂CCl₂CCl₂CCl₂CCl₂CCl₂CCl₂CCl₃ |
| A-164 | CH(CH₃)CH₂CH₂CH₂CH₂CH₂CH₂F |
| A-165 | CH(CH₃)CH₂CH₂CH₂CH₂CH₂CH₂Cl |
| A-166 | CH(CH₃)CH₂CH₂CH₂CH₂CH₂CH₂Br |
| A-167 | CH(CH₃)CH₂CH₂CH₂CH₂CH₂CF₃ |
| A-168 | CHFCH₂CH₂CH₂CH₂CH₂CH₂CH₂CH₃ |
| A-169 | CHClCH₂CH₂CH₂CH₂CH₂CH₂CH₂CH₃ |
| A-170 | CH₂CHFCH₂CH₂CH₂CH₂CH₂CH₂CH₃ |
| A-171 | CH₂CHClCH₂CH₂CH₂CH₂CH₂CH₂CH₃ |
| A-172 | CH₂CH₂CHFCH₂CH₂CH₂CH₂CH₂CH₃ |
| A-173 | CH₂CH₂CHClCH₂CH₂CH₂CH₂CH₂CH₃ |
| A-174 | CH₂CH₂CH₂CHFCH₂CH₂CH₂CH₂CH₃ |
| A-175 | CH₂CH₂CH₂CHClCH₂CH₂CH₂CH₂CH₃ |
| A-176 | CH₂CH₂CH₂CH₂CH₂CHFCH₂CH₂CH₃ |
| A-177 | CH₂CH₂CH₂CH₂CH₂CHClCH₂CH₂CH₃ |
| A-178 | CH₂CH₂CH₂CH₂CH₂CH₂CHFCH₂CH₃ |
| A-179 | CH₂CH₂CH₂CH₂CH₂CH₂CHClCH₂CH₃ |
| A-180 | CH₂CH₂CH₂CH₂CH₂CH₂CH₂CHFCH₃ |
| A-181 | CH₂CH₂CH₂CH₂CH₂CH₂CH₂CHClCH₃ |
| A-182 | CH₂CH₂CH₂CH₂CH₂CH₂CH₂CH₂CH₂F |
| A-183 | CH₂CH₂CH₂CH₂CH₂CH₂CH₂CH₂CH₂Cl |
| A-184 | CH₂CH₂CH₂CH₂CH₂CH₂CH₂CH₂CH₂Br |
| A-185 | CCl₂CH₂CH₂CH₂CH₂CH₂CH₂CH₂CH₃ |
| A-186 | CF₂CH₂CH₂CH₂CH₂CH₂CH₂CH₂CH₃ |
| A-187 | CH₂CH₂CH₂CH₂CH₂CH₂CH₂CH₂CHF₂ |
| A-188 | CH₂CH₂CH₂CH₂CH₂CH₂CH₂CH₂CHCl₂ |
| A-189 | CH₂CH₂CH₂CH₂CH₂CH₂CH₂CH₂CF₃ |
| A-190 | CH₂CH₂CH₂CH₂CH₂CH₂CH₂CH₂CCl₃ |
| A-191 | CF₂CF₂CF₂CF₂CF₂CF₂CF₂CF₂CF₃ |
| A-192 | CCl₂CCl₂CCl₂CCl₂CCl₂CCl₂CCl₂CCl₂CCl₃ |
| A-193 | CH(CH₃)CH₂CH₂CH₂CH₂CH₂CH₂CH₂F |
| A-194 | CH(CH₃)CH₂CH₂CH₂CH₂CH₂CH₂CH₂Cl |
| A-195 | CH(CH₃)CH₂CH₂CH₂CH₂CH₂CH₂CH₂Br |
| A-196 | CH(CH₃)CH₂CH₂CH₂CH₂CH₂CH₂CF₃ |
| A-197 | CHFCH₂CH₂CH₂CH₂CH₂CH₂CH₂CH₂CH₃ |
| A-198 | CHClCH₂CH₂CH₂CH₂CH₂CH₂CH₂CH₂CH₃ |
| A-199 | CH₂CHFCH₂CH₂CH₂CH₂CH₂CH₂CH₂CH₃ |
| A-200 | CH₂CHClCH₂CH₂CH₂CH₂CH₂CH₂CH₂CH₃ |
| A-201 | CH₂CH₂CHFCH₂CH₂CH₂CH₂CH₂CH₂CH₃ |
| A-202 | CH₂CH₂CHClCH₂CH₂CH₂CH₂CH₂CH₂CH₃ |
| A-203 | CH₂CH₂CH₂CHFCH₂CH₂CH₂CH₂CH₂CH₃ |
| A-204 | CH₂CH₂CH₂CHClCH₂CH₂CH₂CH₂CH₂CH₃ |
| A-205 | CH₂CH₂CH₂CH₂CHFCH₂CH₂CH₂CH₂CH₃ |
| A-206 | CH₂CH₂CH₂CH₂CHClCH₂CH₂CH₂CH₂CH₃ |
| A-207 | CH₂CH₂CH₂CH₂CH₂CH₂CHFCH₂CH₂CH₃ |
| A-208 | CH₂CH₂CH₂CH₂CH₂CH₂CHClCH₂CH₂CH₃ |
| A-209 | CH₂CH₂CH₂CH₂CH₂CH₂CH₂CHFCH₂CH₃ |
| A-210 | CH₂CH₂CH₂CH₂CH₂CH₂CH₂CHClCH₂CH₃ |
| A-211 | CH₂CH₂CH₂CH₂CH₂CH₂CH₂CH₂CHFCH₃ |
| A-212 | CH₂CH₂CH₂CH₂CH₂CH₂CH₂CH₂CHClCH₃ |
| A-213 | CH₂CH₂CH₂CH₂CH₂CH₂CH₂CH₂CH₂CH₂F |
| A-214 | CH₂CH₂CH₂CH₂CH₂CH₂CH₂CH₂CH₂CH₂Br |
| A-215 | CH₂CH₂CH₂CH₂CH₂CH₂CH₂CH₂CH₂CH₂Cl |
| A-216 | CCl₂CH₂CH₂CH₂CH₂CH₂CH₂CH₂CH₂CH₃ |
| A-217 | CF₂CH₂CH₂CH₂CH₂CH₂CH₂CH₂CH₂CH₃ |
| A-218 | CH₂CH₂CH₂CH₂CH₂CH₂CH₂CH₂CH₂CHF₂ |
| A-219 | CH₂CH₂CH₂CH₂CH₂CH₂CH₂CH₂CH₂CHCl₂ |
| A-220 | CH₂CH₂CH₂CH₂CH₂CH₂CH₂CH₂CH₂CF₃ |
| A-221 | CH₂CH₂CH₂CH₂CH₂CH₂CH₂CH₂CH₂CCl₃ |
| A-222 | CF₂CF₂CF₂CF₂CF₂CF₂CF₂CF₂CF₂CF₃ |
| A-223 | CCl₂CCl₂CCl₂CCl₂CCl₂CCl₂CCl₂CCl₂CCl₂CCl₃ |
| A-224 | CH(CH₃)CH₂CH₂CH₂CH₂CH₂CH₂CH₂CH₂F |
| A-225 | CH(CH₃)CH₂CH₂CH₂CH₂CH₂CH₂CH₂CH₂Cl |
| A-226 | CH(CH₃)CH₂CH₂CH₂CH₂CH₂CH₂CH₂CH₂Br |
| A-227 | CH(CH₃)CH₂CH₂CH₂CH₂CH₂CH₂CH₂CF₃ |
| A-228 | CH═CH₂ |
| A-229 | CH₂CH═CH₂ |
| A-230 | CH═CHCH₃ |
| A-231 | C(CH₃)═CH₂ |
| A-232 | CH₂CH₂CH═CH₂ |
| A-233 | CH₂CH═CHCH₃ |
| A-234 | CH═CHCH₂CH₃ |
| A-235 | CH(CH₃)CH═CH₂ |
| A-236 | C(CH₃)═CHCH₃ |
| A-237 | CH═C(CH₃)₂ |
| A-238 | CH₂CH₂CH₂CH═CH₂ |
| A-239 | CH₂CH₂CH═CHCH₃ |
| A-240 | CH₂CH═CHCH₂CH₃ |
| A-241 | CH═CHCH₂CH₂CH₃ |
| A-242 | CH(CH₃)CH₂CH═CH₂ |
| A-243 | CH₂C(CH₃)═CHCH₃ |
| A-244 | CH₂CH═C(CH₃)₂ |
| A-245 | CH₂CH₂CH₂CH₂CH═CH₂ |
| A-246 | CH₂CH₂CH₂CH═CHCH₃ |
| A-247 | CH₂CH₂CH═CHCH₂CH₃ |
| A-248 | CH₂CH═CHCH₂CH₂CH₃ |
| A-249 | CH═CHCH₂CH₂CH₂CH₃ |
| A-250 | CH(CH₃)CH₂CH₂CH═CH₂ |
| A-251 | CH(CH₃)CH₂CH═CHCH₃ |
| A-252 | CH₂C(CH₃)═CHCH₂CH₃ |
| A-253 | CH₂CH₂CH═C(CH₃)₂ |
| A-254 | CH₂CH₂CH₂CH₂CH₂CH═CH₂ |
| A-255 | CH₂CH₂CH₂CH₂CH═CHCH₃ |
| A-256 | CH₂CH₂CH₂CH═CHCH₂CH₃ |
| A-257 | CH₂CH₂CH═CHCH₂CH₂CH₃ |
| A-258 | CH₂CH═CHCH₂CH₂CH₂CH₃ |
| A-259 | CH═CHCH₂CH₂CH₂CH₂CH₃ |
| A-260 | CH(CH₃)CH₂CH₂CH₂CH═CH₂ |
| A-261 | CH(CH₃)CH₂CH₂CH═CHCH₃ |
| A-262 | C(CH₃)═CHCH₂CH₂CH₂CH₃ |
| A-263 | CH₂CH₂CH₂CH═C(CH₃)₂ |
| A-264 | CH₂CH₂CH₂CH₂CH₂CH₂CH═CH₂ |
| A-265 | CH₂CH₂CH₂CH₂CH₂CH═CHCH₃ |
| A-266 | CH₂CH₂CH₂CH₂CH═CHCH₂CH₃ |
| A-267 | CH₂CH₂CH₂CH═CHCH₂CH₂CH₃ |
| A-268 | CH₂CH₂CH═CHCH₂CH₂CH₂CH₃ |
| A-269 | CH₂CH═CHCH₂CH₂CH₂CH₂CH₃ |
| A-270 | CH═CHCH₂CH₂CH₂CH₂CH₂CH₃ |
| A-271 | CH(CH₃)CH₂CH₂CH₂CH₂CH═CH₂ |
| A-272 | CH(CH₃)CH₂CH₂CH₂CH═CHCH₃ |
| A-273 | C(CH₃)═CHCH₂CH₂CH₂CH₂CH₃ |
| A-274 | CH₂CH₂CH₂CH₂CH═C(CH₃)₂ |
| A-275 | CH₂CH₂CH₂CH₂CH₂CH₂CH₂CH═CH₂ |
| A-276 | CH₂CH₂CH₂CH₂CH₂CH₂CH═CHCH₃ |
| A-277 | CH₂CH₂CH₂CH₂CH₂CH═CHCH₂CH₃ |
| A-278 | CH₂CH₂CH₂CH₂CH═CHCH₂CH₂CH₃ |
| A-279 | CH₂CH₂CH₂CH═CHCH₂CH₂CH₂CH₃ |
| A-280 | CH₂CH₂CH═CHCH₂CH₂CH₂CH₂CH₃ |
| A-281 | CH₂CH═CHCH₂CH₂CH₂CH₂CH₂CH₃ |
| A-282 | CH═CHCH₂CH₂CH₂CH₂CH₂CH₂CH₃ |
| A-283 | CH(CH₃)CH₂CH₂CH₂CH₂CH₂CH═CH₂ |
| A-284 | CH(CH₃)CH₂CH₂CH₂CH₂CH═CHCH₃ |
| A-285 | C(CH₃)═CHCH₂CH₂CH₂CH₂CH₂CH₃ |
| A-286 | CH₂CH₂CH₂CH₂CH₂CH═C(CH₃)₂ |
| A-287 | CH₂CH₂CH₂CH₂CH₂CH₂CH₂CH₂CH═CH₂ |
| A-288 | CH₂CH₂CH₂CH₂CH₂CH₂CH₂CH═CHCH₃ |
| A-289 | CH₂CH₂CH₂CH₂CH₂CH₂CH═CHCH₂CH₃ |
| A-290 | CH₂CH₂CH₂CH₂CH₂CH═CHCH₂CH₂CH₃ |
| A-291 | CH₂CH₂CH₂CH₂CH═CHCH₂CH₂CH₂CH₃ |
| A-292 | CH₂CH₂CH₂CH═CHCH₂CH₂CH₂CH₂CH₃ |
| A-293 | CH₂CH₂CH═CHCH₂CH₂CH₂CH₂CH₂CH₃ |
| A-294 | CH₂CH═CHCH₂CH₂CH₂CH₂CH₂CH₂CH₃ |
| A-295 | CH═CHCH₂CH₂CH₂CH₂CH₂CH₂CH₂CH₃ |
| A-296 | CH(CH₃)CH₂CH₂CH₂CH₂CH₂CH₂CH═CH₂ |
| A-297 | CH(CH₃)CH₂CH₂CH₂CH₂CH₂CH═CHCH₃ |
| A-298 | C(CH₃)═CHCH₂CH₂CH₂CH₂CH₂CH₂CH₃ |
| A-299 | CH₂CH₂CH₂CH₂CH₂CH₂CH═C(CH₃)₂ |
| A-300 | C≡CH |
| A-301 | CH₂C≡CH |
| A-302 | C≡CCH₃ |
| A-303 | CH₂CH₂C≡CH |
| A-304 | CH₂C≡CCH₃ |
| A-305 | C≡CCH₂CH₃ |
| A-306 | CH(CH₃)C≡CH |
| A-307 | CH₂CH₂CH₂C≡CH |
| A-308 | CH₂CH₂C≡CCH₃ |
| A-309 | CH₂C≡CCH₂CH₃ |
| A-310 | C≡CCH₂CH₂CH₃ |
| A-311 | CH(CH₃)CH₂C≡CH |
| A-312 | CH₂CH₂CH₂CH₂C≡CH |
| A-313 | CH₂CH₂CH₂C≡CCH₃ |
| A-314 | CH₂CH₂C≡CCH₂CH₃ |
| A-315 | CH₂C≡CCH₂CH₂CH₃ |
| A-316 | C≡CCH₂CH₂CH₂CH₃ |
| A-317 | CH(CH₃)CH₂CH₂C≡CH |
| A-318 | CH(CH₃)CH₂C≡CCH₃ |
| A-319 | CH₂CH₂CH₂CH₂CH₂C≡CH |
| A-320 | CH₂CH₂CH₂CH₂C≡CCH₃ |
| A-321 | CH₂CH₂CH₂C≡CCH₂CH₃ |
| A-322 | CH₂CH₂C≡CCH₂CH₂CH₃ |
| A-323 | CH₂C≡CCH₂CH₂CH₂CH₃ |
| A-324 | C≡CCH₂CH₂CH₂CH₂CH₃ |
| A-325 | CH(CH₃)CH₂CH₂CH₂C≡CH |
| A-326 | CH(CH₃)CH₂CH₂C≡CCH₃ |
| A-327 | CH(CH₃)CH₂C≡CCH₂CH₃ |
| A-328 | CH₂CH₂CH₂CH₂CH₂CH₂C≡CH |
| A-329 | CH₂CH₂CH₂CH₂CH₂C≡CCH₃ |
| A-330 | CH₂CH₂CH₂CH₂C≡CCH₂CH₃ |
| A-331 | CH₂CH₂CH₂C≡CCH₂CH₂CH₃ |
| A-332 | CH₂CH₂C≡CCH₂CH₂CH₂CH₃ |
| A-333 | CH₂C≡CCH₂CH₂CH₂CH₂CH₃ |
| A-334 | C≡CCH₂CH₂CH₂CH₂CH₂CH₃ |
| A-335 | CH(CH₃)CH₂CH₂CH₂CH₂C≡CH |
| A-336 | CH(CH₃)CH₂CH₂CH₂C≡CCH₃ |
| A-337 | CH₂CH₂CH₂CH₂CH₂CH₂CH₂C≡CH |
| A-338 | CH₂CH₂CH₂CH₂CH₂CH₂C≡CCH₃ |
| A-339 | CH₂CH₂CH₂CH₂CH₂C≡CCH₂CH₃ |
| A-340 | CH₂CH₂CH₂CH₂C≡CCH₂CH₂CH₃ |
| A-341 | CH₂CH₂CH₂C≡CCH₂CH₂CH₂CH₃ |
| A-342 | CH₂CH₂C≡CCH₂CH₂CH₂CH₂CH₃ |
| A-343 | CH₂C≡CCH₂CH₂CH₂CH₂CH₂CH₃ |
| A-344 | C≡CCH₂CH₂CH₂CH₂CH₂CH₂CH₃ |
| A-345 | CH(CH₃)CH₂CH₂CH₂CH₂CH₂C≡CH |
| A-346 | CH(CH₃)CH₂CH₂CH₂CH₂C≡CCH₃ |
| A-347 | CH₂CH₂CH₂CH₂CH₂CH₂CH₂CH₂C≡CH |
| A-348 | CH₂CH₂CH₂CH₂CH₂CH₂CH₂C≡CCH₃ |
| A-349 | CH₂CH₂CH₂CH₂CH₂CH₂C≡CCH₂CH₃ |
| A-350 | CH₂CH₂CH₂CH₂CH₂C≡CCH₂CH₂CH₃ |
| A-351 | CH₂CH₂CH₂CH₂C≡CCH₂CH₂CH₂CH₃ |
| A-352 | CH₂CH₂CH₂C≡CCH₂CH₂CH₂CH₂CH₃ |
| A-353 | CH₂CH₂C≡CCH₂CH₂CH₂CH₂CH₂CH₃ |
| A-354 | CH₂C≡CCH₂CH₂CH₂CH₂CH₂CH₂CH₃ |
| A-355 | C≡CCH₂CH₂CH₂CH₂CH₂CH₂CH₂CH₃ |
| A-356 | CH(CH₃)CH₂CH₂CH₂CH₂CH₂CH₂C≡CH |
| A-357 | C(CH₃)CH₂CH₂CH₂CH₂CH₂C≡CCH₃ |
| A-358 | CH₂CH₂CH₂CH₂CH₂CN |
| A-359 | CH(CH₃)CH₂CH₂CH₂CN |
| A-360 | CH₂CH(CH₃)CH₂CH₂CN |
| A-361 | CH₂CH₂CH(CH₃)CH₂CN |
| A-362 | CH₂CH₂CH(CH₃)CH₂CN |
| A-363 | CH(CH₃)CH(CH₃)CH₂CN |
| A-364 | CH(CH₃)CH(CH₃)CH₂CN |
| A-365 | CH₂C(CH₃)₂CH₂CN |
| A-366 | CH₂CH₂CH₂CH₂CH₂CH₂CN |
| A-367 | CH(CH₃)CH₂CH₂CH₂CH₂CN |
| A-368 | CH₂CH(CH₃)CH₂CH₂CH₂CN |
| A-369 | CH₂CH₂CH(CH₃)CH₂CH₂CN |
| A-370 | CH₂CH₂CH(CH₃)₂CH₂CH₂CN |
| A-371 | CH₂CH₂CH₂CH(CH₃)CH₂CN |
| A-372 | CH(CH₃)CH(CH₃)CH₂CH₂CN |
| A-373 | CH(CH₃)CH₂CH(CH₃)CH₂CN |
| A-374 | CH₂CH₂C(CH₃)₂CH₂CN |
| A-375 | CH(CH₃)CH₂CH(CH₃)CH₂CN |
| A-376 | CH₂CH₂CH₂CH₂CH₂CH₂CH₂CN |
| A-377 | CH(CH₃)CH₂CH₂CH₂CH₂CH₂CN |
| A-378 | CH₂CH(CH₃)CH₂CH₂CH₂CH₂CN |
| A-379 | CH₂CH₂CH(CH₃)CH₂CH₂CH₂CN |
| A-380 | CH₂CH₂CH₂CH(CH₃)CH₂CH₂CN |
| A-381 | CH₂CH₂CH₂CH₂CH(CH₃)CH₂CN |
| A-382 | CH₂CH₂CH₂CH₂CH(CH₃)CH₂CN |
| A-383 | CH(CH₃)CH(CH₃)CH₂CH₂CH₂CN |
| A-384 | CH₂CH(CH₃)CH(CH₃)CH₂CH₂CN |
| A-385 | CH₂CH₂CH₂C(CH₃)₂CH₂CN |
| A-386 | CH(CH₃)CH₂CH(CH₃)CH₂CH₂CN |
| A-387 | CH₂CH(CH₃)CH(CH₃)CH₂CH₂CN |
| A-388 | CH(CH₃)CH₂CH₂CH(CH₃)CH₂CN |
| A-389 | CH₂CH₂CH₂CH₂CH₂CH₂CH₂CH₂CN |
| A-390 | CH(CH₃)CH₂CH₂CH₂CH₂CH₂CH₂CN |
| A-391 | CH₂CH(CH₃)CH₂CH₂CH₂CH₂CH₂CN |
| A-392 | CH₂CH₂CH(CH₃)CH₂CH₂CH₂CH₂CN |
| A-393 | CH₂CH₂CH₂CH(CH₃)CH₂CH₂CH₂CN |
| A-394 | CH₂CH₂CH₂CH₂CH(CH₃)CH₂CH₂CN |
| A-395 | CH₂CH₂CH₂CH₂CH₂CH(CH₃)CH₂CN |
| A-396 | CH₂CH₂CH₂CH₂C(CH₃)₂CH₂CN |
| A-397 | CH(CH₃)CH(CH₃)CH₂CH₂CH₂CH₂CN |
| A-398 | CH₂CH(CH₃)CH(CH₃)CH₂CH₂CH₂CN |
| A-399 | CH₂CH₂CH₂C(CH₃)₂CH₂CH₂CN |
| A-400 | CH(CH₃)CH₂CH(CH₃)CH₂CH₂CH₂CN |
| A-401 | CH₂CH(CH₃)CH(CH₃)CH₂CH₂CH₂CN |
| A-402 | CH(CH₃)CH₂CH₂CH(CH₃)CH₂CH₂CN |
| A-403 | CH(CH₃)CH₂CH₂CH₂CH(CH₃)CH₂CN |
| A-404 | CH₂CH₂CH₂CH₂CH₂CH₂CH₂CH₂CH₂CN |
| A-405 | CH(CH₃)CH₂CH₂CH₂CH₂CH₂CH₂CH₂CN |
| A-406 | CH₂CH(CH₃)CH₂CH₂CH₂CH₂CH₂CH₂CN |
| A-407 | CH₂CH₂CH(CH₃)CH₂CH₂CH₂CH₂CH₂CN |
| A-408 | CH₂CH₂CH₂CH(CH₃)CH₂CH₂CH₂CH₂CN |
| A-409 | CH₂CH₂CH₂CH₂CH(CH₃)CH₂CH₂CH₂CN |
| A-410 | CH₂CH₂CH₂CH₂CH₂CH₂C(CH₃)₂CH₂CN |
| A-411 | CH(CH₃)CH(CH₃)CH₂CH₂CH₂CH₂CH₂CN |
| A-412 | CH₂CH(CH₃)CH(CH₃)CH₂CH₂CH₂CH₂CN |
| A-413 | CH₂CH₂CH₂C(CH₃)₂CH₂CH₂CH₂CN |
| A-414 | CH(CH₃)CH₂CH(CH₃)CH₂CH₂CH₂CH₂CN |
| A-415 | CH₂CH(CH₃)CH(CH₃)CH₂CH₂CH₂CH₂CN |
| A-416 | CH(CH₃)CH₂CH₂CH(CH₃)CH₂CH₂CH₂CN |
| A-417 | CH(CH₃)CH₂CH₂CH₂C(CH₃)₂CH₂CN |
| A-418 | CH₂CH(CH₃)CH₂CH₂CH(CH₃)₂CH₂CN |
| A-419 | CH(CH₃)CH₂CH₂CH₂CH₂CH(CH₃)CH₂CN |
| A-420 | CH₂CH(CH₃)CH₂CH₂CH₂CH(CH₃)CH₂CN |
| A-421 | CH₂CH₂CH₂CH₂CH₂CH₂CH₂CH₂CH₂CH₂CN |
| A-422 | CH(CH₃)CH₂CH₂CH₂CH₂CH₂CH₂CH₂CH₂CN |
| A-423 | CH₂CH(CH₃)CH₂CH₂CH₂CH₂CH₂CH₂CH₂CN |
| A-424 | CH₂CH₂CH(CH₃)CH₂CH₂CH₂CH₂CH₂CH₂CN |
| A-425 | CH₂CH₂CH(CH₃)CH₂CH₂CH₂CH₂CH₂CN |
| A-426 | CH₂CH₂CH₂CH(CH₃)CH₂CH₂CH₂CH₂CN |
| A-427 | CH₂CH₂CH₂CH₂CH₂C(CH₃)₂CH₂CN |
| A-428 | CH(CH₃)CH(CH₃)CH₂CH₂CH₂CH₂CH₂CH₂CN |
| A-429 | CH₂CH(CH₃)CH(CH₃) CH₂CH₂CH₂CH₂CH₂CN |
| A-430 | CH₂CH₂CH₂C(CH₃)₂CH₂CH₂CH₂CH₂CN |
| A-431 | CH(CH₃)CH₂CH(CH₃)CH₂CH₂CH₂CH₂CH₂CN |
| A-432 | CH₂CH(CH₃)CH(CH₃)CH₂CH₂CH₂CH₂CH₂CN |
| A-433 | CH(CH₃)CH₂CH₂CH(CH₃)CH₂CH₂CH₂CH₂CN |
| A-434 | CH(CH₃)CH₂CH₂CH₂CH(CH₃)CH₂CH₂CH₂CN |
| A-435 | CH(CH₃)CH₂CH₂CH₂CH₂CH(CH₃)CH₂CH₂CN |
| A-436 | CH(CH₃)CH₂CH₂CH₂CH₂CH₂CH(CH₃)CH₂CN |
| A-437 | CH(CH₃)CH₂CH₂CH₂CH₂CH₂C(CH₃)CH₂CN |
| A-438 | CH₂CH(CH₃)CH₂CH₂CH₂CH₂CH(CH₃)CH₂CN |
| A-439 | CH(CH₃)CH₂CH₂CH₂CH₂C(CH₃)₂CH₂CN |
| A-440 | CH₂CH(CH₃)CH₂CH₂CH₂C(CH₃)₂CH₂CN |
| A-441 | CH₂CH₂CH₂CH₂CH₂CH₂CH₂CH₂CH₂CH₂CH₂CN |
| A-442 | CH(CH₃)CH₂CH₂CH₂CH₂CH₂CH₂CH₂CH₂CH₂CN |
| A-443 | CH₂CH(CH₃)CH₂CH₂CH₂CH₂CH₂CH₂CH₂CH₂CN |
| A-444 | CH₂CH₂CH(CH₃)CH₂CH₂CH₂CH₂CH₂CH₂CH₂CN |
| A-445 | CH₂CH₂CH₂CH(CH₃)CH₂CH₂CH₂CH₂CH₂CH₂CN |
| A-446 | CH₂CH₂CH₂CH₂CH(CH₃)CH₂CH₂CH₂CH₂CH₂CN |
| A-447 | CH₂CH₂CH₂CH₂CH₂CH₂CH₂C(CH₃)₂CH₂CN |
| A-448 | CH(CH₃)CH(CH₃)CH₂CH₂CH₂CH₂CH₂CH₂CH₂CN |
| A-449 | CH₂CH(CH₃)CH(CH₃)CH₂CH₂CH₂CH₂CH₂CH₂CN |
| A-450 | CH₂CH₂CH₂C(CH₃)₂CH₂CH₂CH₂CH₂CH₂CN |
| A-451 | CH(CH₃)CH₂CH(CH₃)CH₂CH₂CH₂CH₂CH₂CH₂CN |
| A-452 | CH₂CH(CH₃)CH(CH₃)CH₂CH₂CH₂CH₂CH₂CH₂CN |
| A-453 | CH(CH₃)CH₂CH₂CH(CH₃)CH₂CH₂CH₂CH₂CH₂CN |
| A-454 | CH(CH₃)CH₂CH₂CH₂CH(CH₃)CH₂CH₂CH₂CH₂CN |
| A-455 | CH(CH₃)CH₂CH₂CH₂CH₂CH(CH₃)CH₂CH₂CH₂CN |
| A-456 | CH(CH₃)CH₂CH₂CH₂CH₂CH₂CH(CH₃)CH₂CH₂CN |
| A-457 | CH(CH₃)CH₂CH₂CH₂CH₂CH₂CH₂CH(CH₃)CH₂CN |
| A-458 | CH₂CH(CH₃)CH₂CH₂CH₂CH₂CH(CH₃)CH₂CH₂CN |
| A-459 | CH₂CH(CH₃)CH₂CH₂CH₂CH₂CH(CH₃)CH₂CH₂CN |
| A-460 | CH₂CH₂CH(CH₃)CH₂CH₂CH₂CH₂CH(CH₃)CH₂CN |
| A-461 | CH₂CH(CH₃)CH₂CH₂CH₂CH₂C(CH₃)₂CH₂CN |
| A-462 | CH₂CH₂CH₂CH₂CH₂CH₂CH₂CH₂CH₂CH₂CH₂CH₂CN |
| A-463 | CH(CH₃)CH₂CH₂CH₂CH₂CH₂CH₂CH₂CH₂CH₂CH₂CN |
| A-464 | CH₂CH(CH₃)CH₂CH₂CH₂CH₂CH₂CH₂CH₂CH₂CH₂CN |
| A-465 | CH₂CH₂CH(CH₃)CH₂CH₂CH₂CH₂CH₂CH₂CH₂CH₂CN |
| A-466 | CH₂CH₂CH₂CH(CH₃)CH₂CH₂CH₂CH₂CH₂CH₂CH₂CN |
| A-467 | CH₂CH₂CH₂CH₂CH(CH₃)CH₂CH₂CH₂CH₂CH₂CH₂CN |
| A-468 | CH₂CH₂CH₂CH₂CH₂CH(CH₃)CH₂CH₂CH₂CH₂CH₂CN |
| A-469 | CH₂CH₂CH₂CH₂CH₂CH₂CH(CH₃)CH₂CH₂CH₂CH₂CN |
| A-470 | CH₂CH₂CH₂CH₂CH₂CH₂CH₂CH(CH₃)CH₂CH₂CH₂CN |
| A-471 | CH₂CH₂CH₂CH₂CH₂CH₂CH₂CH₂CH(CH₃)CH₂CH₂CN |
| A-472 | CH₂CH₂CH₂CH₂CH₂CH₂CH₂CH₂CH₂CH(CH₃)CH₂CN |
| A-473 | CH(CH₃)CH(CH₃)CH₂CH₂CH₂CH₂CH₂CH₂CH₂CH₂CN |
| A-474 | CH₂CH(CH₃)CH(CH₃)CH₂CH₂CH₂CH₂CH₂CH₂CH₂CN |
| A-475 | CH₂CH₂CH₂C(CH₃)₂CH₂CH₂CH₂CH₂CH₂CH₂CN |
| A-476 | CH₂CH₂CH₂CH₂CH(CH₃)CH₂CH₂CH₂CH₂CH₂CH₂CN |
| A-477 | CH(CH₃)CH₂CH(CH₃)CH₂CH₂CH₂CH₂CH₂CH₂CH₂CN |
| A-478 | CH(CH₃)CH₂CH₂CH(CH₃)CH₂CH₂CH₂CH₂CH₂CH₂CN |
| A-479 | CH(CH₃)CH₂CH₂CH₂CH(CH₃)CH₂CH₂CH₂CH₂CH₂CN |
| A-480 | CH(CH₃)CH₂CH₂CH₂CH₂CH(CH₃)CH₂CH₂CH₂CH₂CN |
| A-481 | CH(CH₃)CH₂CH₂CH₂CH₂CH₂CH(CH₃)CH₂CH₂CH₂CN |
| A-482 | CH(CH₃)CH₂CH₂CH₂CH₂CH₂CH₂CH(CH₃)CH₂CH₂CN |
| A-483 | CH₂CH(CH₃)CH₂CH₂CH₂CH₂CH(CH₃)CH₂CH₂CH₂CN |
| A-484 | CH₂CH₂CH(CH₃)CH₂CH₂CH₂CH₂CH(CH₃)CH₂CH₂CN |
| A-485 | CH₂CH₂CH₂CH(CH₃)CH₂CH₂CH₂CH(CH₃)CH₂CH₂CN |
| A-486 | CH₂CH(CH₃)CH₂CH₂CH₂CH₂CH₂C(CH₃)₂CH₂CN |
| A-487 | CHFCH₂CN |
| A-488 | CHClCH₂CN |
| A-489 | CCl₂CH₂CN |
| A-490 | CF₂CH₂CN |
| A-491 | CHFCH₂CH₂CN |
| A-492 | CHClCH₂CH₂CN |
| A-493 | CCl₂CH₂CH₂CN |
| A-494 | CF₂CH₂CH₂CN |
| A-495 | CHFCH₂CH₂CH₂CN |
| A-496 | CHClCH₂CH₂CH₂CN |
| A-497 | CCl₂CH₂CH₂CH₂CN |
| A-498 | CF₂CH₂CH₂CH₂CN |
| A-499 | CHFCH₂CH₂CH₂CH₂CN |
| A-500 | CHClCH₂CH₂CH₂CH₂CN |
| A-501 | CCl₂CH₂CH₂CH₂CH₂CN |
| A-502 | CF₂CH₂CH₂CH₂CH₂CN |
| A-503 | CHFCH₂CH₂CH₂CH₂CH₂CN |
| A-504 | CHClCH₂CH₂CH₂CH₂CH₂CN |
| A-505 | CCl₂CH₂CH₂CH₂CH₂CH₂CN |
| A-506 | CF₂CH₂CH₂CH₂CH₂CH₂CN |
| A-507 | CHFCH₂CH₂CH₂CH₂CH₂CH₂CN |
| A-508 | CHClCH₂CH₂CH₂CH₂CH₂CH₂CN |
| A-509 | CCl₂CH₂CH₂CH₂CH₂CH₂CH₂CN |
| A-510 | CF₂CH₂CH₂CH₂CH₂CH₂CH₂CN |
| A-511 | CHFCH₂CH₂CH₂CH₂CH₂CH₂CH₂CN |
| A-512 | CHClCH₂CH₂CH₂CH₂CH₂CH₂CH₂CN |
| A-513 | CCl₂CH₂CH₂CH₂CH₂CH₂CH₂CH₂CN |
| A-514 | CF₂CH₂CH₂CH₂CH₂CH₂CH₂CH₂CN |
| A-515 | CHFCH₂CH₂CH₂CH₂CH₂CH₂CH₂CH₂CN |
| A-516 | CHClCH₂CH₂CH₂CH₂CH₂CH₂CH₂CH₂CN |
| A-517 | CCl₂CH₂CH₂CH₂CH₂CH₂CH₂CH₂CH₂CN |
| A-518 | CF₂CH₂CH₂CH₂CH₂CH₂CH₂CH₂CH₂CN |
| A-519 | CHFCH₂CH₂CH₂CH₂CH₂CH₂CH₂CH₂CH₂CN |
| A-520 | CHClCH₂CH₂CH₂CH₂CH₂CH₂CH₂CH₂CH₂CN |
| A-521 | CCl₂CH₂CH₂CH₂CH₂CH₂CH₂CH₂CH₂CH₂CN |
| A-522 | CF₂CH₂CH₂CH₂CH₂CH₂CH₂CH₂CH₂CH₂CN |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes, in particular from the class of the Oomycetes. Some are systemically effective and they can be used in plant protection as foliar fungicides, as fungicides for seed dressing and soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Bremia lactucae on lettuce,
Erysiphe cichoracearum and Sphaerotheca fuliginea on cucurbits,
Fusarium and Verticillium species on various plants,
Mycosphaerella species on cereals, bananas and peanuts,
Peronospora species on cabbage and bulbous plants,
Phakopsora pachyrhizi and P. meibomiae on soybeans
Phytophthora infestans on potatoes and tomatoes,
Phytophthora capsici on peppers,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Pythium aphanidermatum on lawns,
Rhizoctonia species on cotton, rice and lawns,
Septoria tritici and Stagonospora nodorum on wheat,
Uncinula necatoron grapevines,
Ustilago species on cereals and sugar cane, and
Venturia species (scab) on apples and pears.

They are particularly suitable for controlling harmful fungi from the class of the Oomycetes, such as Peronospora species, Phytophthora species, Plasmopara viticola and Pseudoperonospora species.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 1 to 1000 g/100 kg, preferably 5 to 100 g/100 kg of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations:

### 1. Products for Dilution With Water

### A Water-Soluble Concentrates (SL)

10 parts by weight of a compound according to the invention are dissolved in water or in a water-soluble solvent. As an alternative, wetters or other auxiliaries are added. The active compound dissolves upon dilution with water.

### B Dispersible Concentrates (DC)

20 parts by weight of a compound according to the invention are dissolved in cyclohexanone with addition of a dispersant, for example polyvinylpyrrolidone. Dilution with water gives a dispersion.

### C Emulsifiable Concentrates (EC)

15 parts by weight of a compound according to the invention are dissolved in xylene with addition of calcium dodecylbenzenesulfonate and castor oil ethoxylate (in each case 5%). Dilution with water gives an emulsion.

### D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

### E Suspensions (SC, OD)

In an agitated ball mill, 20-parts by weight of a compound according to the invention are comminuted with addition of dispersants, wetters and water or an organic solvent to give a fine active compound suspension. Dilution with water gives a stable suspension of the active compound.

### F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.

### G Water-Dispersible Powders and Water-Soluble Powders (WP, SP)

75 parts by weight of a compound according to the invention are ground in a rotor-stator mill with addition of dispersants, wetters and silica gel. Dilution with water gives a stable dispersion or solution of the active compound.

### 2. Products to be Applied Undiluted

### H Dustable Powders (DP)

5 parts by weight of a compound according to the invention are ground finely and mixed intimately with 95% of finely divided kaolin. This gives a dustable product.

### I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.

### J ULV Solutions (UL)

10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the application form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, enilconazole, epoxiconazole, fenbuconazole, fluquinconazole, flusilazole, flutriafol, hexaconazole, imazalil, ipconazole, metconazole, myclobutanil, penconazole, propiconazole, prochloraz, prothioconazole, simeconazole, tebuconazole, tetraconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, picobenzamid, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthalisopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, diclomezine, diclocymet, diethofencarb, edifenphos, ethaboxam, fenhexamid, fentin acetate, fenoxanil, ferimzone, fluazinam, fosetyl, phosphorous acid, fosetyl-aluminum, iprovalicarb, hexachlorobenzene, metrafenone, pencycuron, propamocarb, phthalide, tolclofosmethyl, quintozene or zoxamide,
strobilurins, such as azoxystrobin, dimoxystrobin, enestroburin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures given in the synthesis examples below were, with appropriate modification of the starting materials, used to obtain further compounds I. The compounds obtained in this manner are listed in the table that follows, together with physical data.

### EXAMPLE 1

### Preparation of 6-(3-Bromopropyl)-5-ethyl-[1,2,4]triazolo[1,5-a]pyrimidin-7-ylamine [I-1]

At 20 to 25° C., 0.60 ml of 48% strength aqueous hydrobromic acid was added to a solution of 495 mg (1.7 mmol) of 5-ethyl-6-(3-pentyloxypropyl)-[1,2,4]triazolo-[1,5-a]pyrimidin-7-ylamine (preparation analogously to EP-A 141 317) in 5 ml of glacial acetic acid, and the mixture was then heated under reflux for 20 hours. After cooling, the volatile components were removed from the reaction mixture, the residue was taken up in CH₂Cl₂/H₂O and the aqueous phase was washed with saturated NaHCO₃ solution until neutral. The organic phase was separated off, washed with water and dried, and the solvent was removed. The residue gave, after chromatography on RP18 phase (MPLC isocratic; acetonitrile/water mixture), 0.21 g of the title compound in the form of white crystals.

### EXAMPLE 2

### Preparation of 7-Amino-6-(5-cyanopentyl)-5-ethyl-[1,2,4]triazolo-[1,5-a]pyrimidine

### 2.a) 4,9-Dicyanononan-3-one

5.6 g of ethyl propionate were added dropwise to a solution of 6.8 g of 1,6-dicyano-hexane and 11.2 g of 95% pure potassium tert-butoxide in 100 ml of anhydrous dimethylformamide (DMF). After the addition had ended, the reaction mixture was stirred at 20 to 25° C. for 17 hours and then diluted with water and washed with tert-butyl methyl ether (MTBE). After acidification with concentrated HCl, the aqueous phase was extracted with MTBE. This ether phase was washed with water and, after drying, freed from the solvent. What remained were 7.1 g of the title compound as an oil which was reacted without further purification.

### 2.b) 7-Amino-6-(5-cyanopentyl)-5-ethyltriazolo-(1,5-a)-pyrimidine [1-3]

4.76 g of 4,9-dicyanononan-3-one, 2.5 g of 3-amino-1H-1,2,4-triazole and 0.94 g of p-toluenesulfonic acid in 25 ml of mesitylene were stirred at 170° C. for four hours, during which time small amounts of mesitylene were distilled off continuously. The solvent was then distilled off, and the residue was taken up in dichloromethane and water. After removal of insoluble components, the organic phase was washed with water, saturated NaHCO₃ solution and saturated NaCl solution and then dried, and volatile components were removed. The residue was digested with MTBE. After removal of the solvent, 2.0 g of the title compound remained as colorless crystals of m.p. 158-160° C.

### EXAMPLE 3

### Preparation of 5-Ethyl-6-(5,6,6-trifluorohex-5-enyl)-[1,2,4]-triazolo[1,5-a]pyrimidin-7-ylamine [1-5]

### 3a) Methyl 7,8,8-trifluoro-2-propionyloct-7-enoate

At 20 to 25° C., 5.40 g of methanolic potassium methoxide solution (30% strength, 23 mmol) were added dropwise to a solution of 3.30g (23 mmol) of ethylpropionyl acetate in 2.5 ml of methanol. After 1 hour of stirring at this temperature and then 30 min of stirring at 40° C., 5.00 g (23 mmol) of 6-bromo-1,1,2-trifluoro-1-hexene were added dropwise at 40° C. over a period of 5 min. The reaction mixture was then stirred at this temperature for 15 hours. The suspension formed was taken up in methyl tert-butyl ether (MTBE) and then filtered through silica gel. The eluate was washed with water and then with saturated NaCl solution and then dried, and the solvent was removed. What remained were 2.34 g of the title compound as a colorless oil.

### 3b) 5-Ethyl-6-(5,6,6-trifluorohex-5-enyl)-[1,2,4]triazolo[1,5-a]pyrimidin-7-ol

A mixture of 5.28 mmol of methyl 7,8,8-trifluoro-2-propionyloct-7-enoate, 0.86 g (10.2 mmol) of 3-amino-1,2,4-triazole and 10 ml of propionic acid was heated under reflux for about 15 hours. The propionic acid was then distilled off, and the residue was chromatographed on silica gel (cyclohexane/ethyl acetate mixture). What remained was 0.6 g of the title compound in the form of yellow crystals.

### 3c) 7-Chloro-5-ethyl-6-(5,6,6-trifluorohex-5-enyl)-[1,2,4]triazolo[1,5-a]pyrimidine

0.60 g (2 mmol) of the compound from Ex. 3b) in 20 ml of phosphoryl chloride was heated under reflux for 15 hours. The volatile components were then distilled off, the residue was taken up in CH₂Cl₂, the solution was washed with NaHCO₃ solution until neutral and dried and the solvent was removed. The residue gave, after chromatography on silica gel (ethyl acetate/methanol mixture), 0.38 g of the title compound as a yellow oil.

### 3d) 5-Ethyl-6-(5,6,6-trifluorohex-5-enyl)-[1,2,4]triazolo[1,5-a]pyrimidin-7-ylamine

A solution of 0.35 g (1.1 mmol) of the compound from Ex. 3c) in 2 ml of methanol and 10 ml of a 7M methanolic NH₃ solution were stirred at 20 to 25° C. for 48 hours. The solution was freed from the volatile components and the residue was suspended in water in an ultrasonic bath, filtered off and then dried. What remained was 0.21 g of the title compound in the form of white crystals of m.p. 199° C.

TABLE I
Compounds of the formula I

| | | | Phys. data (m.p. [° C.]; |
|---|---|---|---|
| No. | R¹ | R² | ¹H-NMR δ [ppm]) |
| I-1 | CH₂CH₂CH₂Br | CH₂CH₃ | 240-241 |
| I-2 | CH₂CH₂CH₂Cl | CH₂CH₃ | 8.4 (s, 1H), 7.8 (s, 2H), |
| | | | 3.7 (t, 2H), 2.8 (q, 2H), 2.7 (m, |
| | | | 2H), 1.9 (m, 2H), 1.2 (t, 3H). |
| I-3 | (CH₂)₅CN | CH₂CH₃ | 158-160 |
| I-4 | (CH₂)₅CN | CH₂CH₂CH₃ | 158 |
| I-5 | (CH₂)₄CH═CH₂ | CH₂CH₃ | 199 |
| I-6 | (CH₂)₄CH═CH₂ | CH₃ | 209-210 |
| I-7 | (CH₂)₄CF═CF₂ | CH₃ | 190-191 |

### Examples of the Action Against Harmful Fungi

The fungicidal action of the compounds of the formula I was demonstrated by the following experiments:

The active compounds were prepared as a stock solution with 25 mg of active compound which was made up to 10 ml with a mixture of acetone and/or DMSO and the emulsifier Uniperol® EL (wetting agent having emulsifying and dispersing action based on ethoxylated alkylphenols) in a ratio by volume of solvent/emulsifier of 99/1. The mixture was then made up to 100 ml with water. This stock solution was, using the solvent/emulsifier/water mixture described, diluted to the active compound concentration stated below.

Use Example 1—Activity against peronospora of grapevines caused by Plasmopara viticola

Leaves of potted vines were sprayed to runoff point with an aqueous suspension having the concentration of active compounds stated below. The next day, the undersides of the leaves were inoculated with an aqueous sporangia suspension of Plasmopara viticola. The vines were then initially placed in a water-vapor-saturated chamber at 24° C. for 48 hours and then in a greenhouse at temperatures between 20 and 30° C. for 5 days. After this time, the plants were once more placed in a humid chamber for 16 hours to promote the eruption of sporangiophores. The extent of the development of the infection on the undersides of the leaves was then determined visually.

In this test, the plants which had been treated with 250 ppm of the compound I-7 showed no infection, whereas the untreated plants were 90% infected.

Use Example 2: Activity against late blight of tomatoes caused by Phytophthora infestans, protective treatment

Leaves of potted tomato plants were sprayed to runoff point with an aqueous suspension of the active compounds. Four days after the application, the leaves were infected with an aqueous sporangia suspension of Phytophthora infestans. The plants were then placed on a water-vapor-saturated chamber at temperatures between 18 and 20° C. After 6 days the infection was determined visually in %.

In this test, the plants which had been treated with 250 ppm of the compound I-7 showed no infection, whereas the untreated plants were 100% infected.

1. A triazolopyrimidine of the formula I 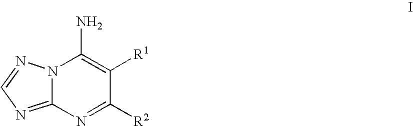
in which the substituents are as defined below:
R¹ is C₁-C₁₂-alkenyl or C₂C₁₂-alkynyl, where the carbon chains are unsubstituted or carry one to three identical or different groups R^{a} and/or R^{b}:
or
C₁-C₁₄alkyl, C₁-C₁₂-alkoxy-C₁-C₁₂alkyl, C₁-C₆-alkoxy-C₂-C₁₂-alkenyl or C₁-C₆-alkoxy-C₂-C₁₂alkynyl, where the carbon chains carry one to three identical or different groups R^{a};
R^{a} is halogen, cyano, nitro, hydroxyl, C₁-C₆-alkylthio, C₃-C₁₂-alkenyloxy, C₃-C₁₂-alkynyloxy, or
C₃C₆-cycloalkyl which may carry one to four identical or different groups R^{b};
R^{b} is C₁-C₄-alkyl, cyano, nitro, hydroxyl, C₁-C₆-alkoxy, C₁-C₆-alkylthio, C₃-C₆-alkenyloxy and C₃-C₆-alkynyloxy;
where the carbon chains of the groups R^{a} for their part may be halogenated;
R² is C₁-C₁₂-alkyl, C₂-C₁₂-alkenyl or C₂-C₁₂-alkynyl, where the carbon chains are substituted by one to three groups R^{c}:
R^{c} is cyano, nitro, hydroxyl; or C₃-C₆-cycloalkyl which may carry one to four identical or different groups C₁-C₄-alkyl, halogen, cyano, nitro, hydroxyl, C₁-C₆-alkoxy, C₁-C₆-alkylthio, C₃-C₆-alkenyloxy or C₃-C₆-alkynyloxy.

2. The compound of the formula I according to claim 1 in which
R¹ is C₁-C₁₄-haloalkyl, C₁-C₁₂-haloalkoxy-C₁-C₁₂-alkyl, C₁-C₁₂-alkoxy-C₁-C₁₂-haloalkyl, C₂-C₁₂-alkenyl, C₂-C₁₂-haloalkenyl, C₂-C₁₂-alkynyl or C₂-C₁₂-haloalkynyl, where the carbon chains may carry one to three groups R^{a}:
R^{a} is cyano, nitro, hydroxyl, C₁-C₆-alkoxy, C₁-C₆-alkylthio, C₃-C₁₂-alkenyloxy, C₃-C₁₂-alkynyloxy, or
C₃-C₆-cycloalkyl which may carry one to four identical or different groups;
R^{b} is C₁-C₄-alkyl, cyano, nitro, hydroxyl, C₁-C₆-alkoxy, C₁-C₆-alkylthio, C₃-C₆-alkenyloxy and C₃-C₆-alkynyloxy
where the carbon chains of the groups R^{a} for their part may be halogenated.

3. The compound of the formula 1 according to claim 1 or 2 in which
R² is C₁-C₁₂-alkyl, C₂-C₁₂-alkenyl or C₂-C₁₂-alkynyl, where the carbon chains may be substituted by one to three groups R^{c}:
R^{c} is cyano, nitro, hydroxyl; or C₃-C₆-cycloalkyl which may carry one to four identical or different groups C₁-C₄-alkyl, halogen, cyano, nitro, hydroxyl, C₁-C₆-alkoxy, C₁-C₆-alkylthio, C₃-C₆-alkenyloxy or C₃-C₆-alkynyloxy.

4. The compound of the formula I according to claim 1 in which
R¹ is C₁-C₁₄-alkyl, where the carbon chains carry one to three identical or different groups cyano or halogen.

5. The compound of the formula I according to claim 1 in which
R¹ is C₂-C₁₂-alkenyl or C₂-C₁₂-alkynyl, where the carbon chains are unsubstituted or carry one to three identical or different groups R^{a} and/or R^{b}.

6. The compound of the formula I according to claim 1 in which R¹ and R² together do not have more than 14 carbon atoms.

7. The compound of the formula I according to claim 1 in which R¹ is chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 1,1,1-trifluoroprop-2-yl, 1-chloropropyl, 1-fluoropropyl, 3-chloropropyl, 3-fluoropropyl, 3,3,3-trifluoropropyl, 1-chlorobutyl, 1-fluorobutyl, 4-chlorobutyl, 4-fluorobutyl, 4,4,4-trifluorobutyl, 1-chloropentyl, 1-fluoropentyl, 5,5,5-trifluoropentyl, 5-chloropentyl, 5-fluoropentyl, 1-chlorohexyl, 1-fluorohexyl, 6-chlorohexyl, 6-fluorohexyl, 6,6,6-trifluorohexyl, 1-chloroheptyl, 1-fluoroheptyl, 7-chloroheptyl, 7-fluoroheptyl, 7,7,7-trifluoroheptyl, 1-chlorooctyl, 1-fluorooctyl, 8-fluorooctyl, 8,8,8-trifluorooctyl, 1-chlorononyl, 1-fluorononyl, 9-fluorononyl, 9,9,9-trifluorononyl, 9-chlorononyl, 1-fluorodecyl, 1-chlorodecyl, 10-fluorodecyl, 10,10,10-trifluorodecyl, 10-chlorodecyl, 1-chloroundecyl, 1-fluoroundecyl, 11-chloroundecyl, 11-fluoroundecyl, 11,11,11-trifluoroundecyl, 1-chlorododecyl, 1-fluorododecyl, 12-chlorododecyl, 12-fluorododecyl or 12,12,12-trifluorododecyl.

8. The compound of the formula I according to claim 1 in which R² is methyl, ethyl, isopropyl, n-propyl or n-butyl.

9. The compound of the formula I according to claim 1:
6-(3-bromopropyl)-5-ethyl-[1,2,4]triazolo[1,5-a]pyrimidin-7-ylamine;
6-(3-chloropropyl)-5-ethyl-[1,2,4]triazolo[1,5-a]pyrimidin-7-ylamine;
6-(7-amino-5-ethyl-[1,2,4]triazolo[1,5-a]pyrimidin-6-yl)-hexanenitrile;
6-(7-amino-5-propyl-[1,2,4]triazolo[1,5-a]pyrimidin-6-yl)-hexanenitrile;
5-ethyl-6-hex-5-enyl-[1,2,4]triazolo[1,5-a]pyrimidin-7-ylamine;
6-hex-5-enyl-5-methyl-[1,2,4]triazolo[1,5-a]pyrimidin-7-ylamine;
5-methyl-6-(5,6,6-trifluorohex-5-enyl)-[1,2,4]triazolo[1,5-a]pyrimidin-7-ylamine.

10. A process for preparing compounds of the formula I according to claim 1 wherein β-ketoesters of the formula II, 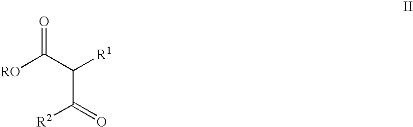
in which R is C₁-C₄-alkyl are reacted with 3-amino-1,2,4-triazole of the formula III 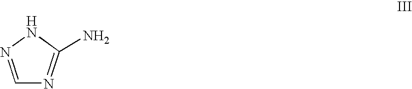
to give 7-hydroxytriazolopyrimidines of the formula IV 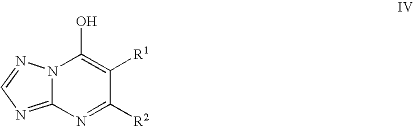
which are halogenated to give compounds of the formula V 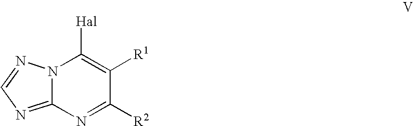
in which Hal is chlorine or bromine and V is reacted with ammonia.

11. A process for preparing compounds of the formula I according to claim 1 wherein acylcyanides of the formula VI, 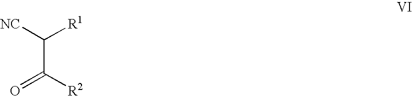
are reacted with 3-amino-1,2-triazole of the formula III.

12. A compound of the formula IV or V according to claim 10.

13. A process for preparing compounds of the formula I according to claim 1 in which R¹ is halogen-substituted C₁-C₁₄-alkyl, C₁-C₁₂-alkoxy-C₁-C₁₂-alkyl, C₂-C₁₂-alkenyl or C₂-C₁₂-alkynyl, by halogenating triazolopyrimidines of the formula VII, 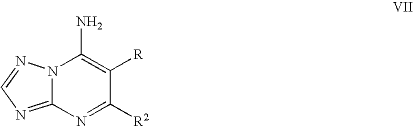
in which R is C₁-C₁₄-alkyl, C₁-C₁₂-alkoxy-C₁-C₁₂-alkyl, C₂-C₁₂-alkenyl, C₂-C₁₂-alkynyl, where the carbon chains may carry one to three groups R^{a} as set forth in claim 1, using a halogenating agent in the presence of a free-radical initiator or an acid.

14. A fungicidal composition comprising a solid or liquid carrier and a compound of the formula I according to claim 1.

15. Seed comprising a compound of the formula I according to claim 1 in an amount of 1 to 1000 g per 100 kg.

16. A method for controlling phytopathogenic harmful fungi wherein the fungi or the materials, plants, the soil or seed to be protected against fungal attack are treated with an effective amount of a compound of the formula I according to claim 1.
